(19) United States
(12) Patent Application Publication
(10) Pub. No.: US 2020/0268641 A1
(43) Pub. Date: Aug. 27, 2020

(54) NAIL ENAMEL COMPOSITION

(71) Applicant: L'ORÉAL, Paris (FR)

(72) Inventors: Jianxin Feng, Clark, NJ (US); Ramakrishnan Hariharan, Springfield, NJ (US)

(73) Assignee: L'ORÉAL, Paris (FR)

(21) Appl. No.: 16/287,358

(22) Filed: Feb. 27, 2019

(51) Int. Cl. A61K 8/92; A61Q 3/02; A61K 8/37; A61K 8/34; A61K 8/49; A61K 8/73; A61K 8/362; A61K 8/368; A61K 8/36

(52) U.S. Cl. CPC A61K 8/922; A61Q 3/02; A61K 8/37; A61K 8/34; A61K 2800/43; A61K 8/731; A61K 8/362; A61K 8/368; A61K 8/361; A61K 8/4973

(57) ABSTRACT

The present disclosure is drawn to nail enamel compositions including cannabis sativa seed oil, and specifically to nail enamel compositions including a plasticizer, a solvent, a film forming polymer, and cannabis sativa seed oil. The plasticizer may be, e.g., a citric acid and/or benzoic acid derivative, and may be present in a total amount of between 1.0% and 10.0% by weight. The solvent may be, e.g., ethyl acetate, propyl acetate, butyl acetate, ethyl lactate, isopropanol, and/or propylene carbonate, and may be present in a total amount of at least 10%. The film forming polymer may include, e.g., a cellulose derivative and/or may include two or more polymers. The film forming polymer may be present in a total amount of between 5.0% and 70.0% by weight. The composition may include a colorant, such as a pigment, and the pigment may be present in an amount of at least 0.5% by weight.

### FIELD OF THE INVENTION

The present invention relates to nail enamel compositions, and specifically to nail enamel compositions including cannabis sativa seed oil as a natural additive to introduce unique performance and properties into regular nail polishes, including improved durability.

### BACKGROUND

Consumers use nail enamel to cosmetically enhance the appearance of their nails or protect the nails from the abuses found in their everyday environment. Often this requires a nail enamel that is formulated to provide a good shine.

Consumers also desire a durable nail enamel. Lack of durability is often evidenced by unsightly chipping or peeling of the coating soon after the original coating has been applied, requiring at least in part a reapplication of the coating in an attempt to recreate the aesthetic appearance or the therapeutic benefits of the original nail coating.

A durable nail enamel is therefore highly desirable.

### BRIEF SUMMARY

In a first aspect, the present invention is directed to a nail composition having (a) a plasticizer, (b) a solvent, (c) a film forming polymer, an (d) cannabis sativa seed oil. Advantageously, the plasticizer includes a citric acid derivative and/or a benzoic acid derivative. Optionally, the plasticizer is present in the composition in an amount of between 1.0% and 10.0% by weight. Advantageously, the solvent includes ethyl acetate, propyl acetate, butyl acetate, ethyl lactate, isopropanol, and/or propylene carbonate. Optionally, the solvent is present in an amount greater than 10% by weight, such as between 10% and 80% by weight.

Advantageously, the cannabis sativa seed oil is present in an amount less than or equal to 10.0% by weight. Advantageously, the film forming polymer is present in a total amount of between 5% and 40% by weight. Advantageously, the film forming polymer includes a cellulose derivative. Optionally, the film forming polymer includes at least two polymers.

Advantageously, the ratio of total amount of film forming polymer to the total amount of cannabis sativa seed oil by weight, is between 5:1 and 40:1.

Optionally, the composition includes at least one pigment, which may be present in an amount greater than 0.5% by weight.

In a second aspect, the present invention is directed to a nail composition having (a) a plasticizer, (b) a solvent, (c) a film forming polymer, an (d) an oil having a ratio of between 2:1 and 4:1 of omega-6 fatty acids to omega-3 fatty acids.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “between [two numbers]” is inclusive of both numbers.

As used herein, the term “at least one” means one or more and thus includes individual components as well as mixtures/combinations.

As used herein, the terms “include”, “includes” and “including” are meant to be non-limiting.

The present invention is directed to a nail composition having (a) a plasticizer, (b) a solvent, (c) a film forming polymer, an (d) an oil having a ratio of between 2:1 and 4:1 of omega-6 fatty acids to omega-3 fatty acids, such as cannabis sativa seed oil. Each of these will be discussed in turn.

According to embodiments of the present invention, the nail composition also includes at least one plasticizer. The plasticizer is preferably selected from the group consisting of a benzoic acid derivative, a citric acid derivative, and an isobutyrate derivative. More preferably, the plasticizer is a benzoic acid derivative and/or a citric acid derivative.

In certain embodiments, the benzoic acid derivative may be a dibenzoate ester, including but not limited to diethylene glycol dibenzoate, dipropylene glycol dibenzoate, and 1,2-propylene glycol dibenzoate.

In certain embodiments, the citric acid derivative may be an optionally hydroxylated triester of a C₂-C₈ tricarboxylic acid and of a C₂-C₈ alcohol, such as citric acid esters, such as trioctyl citrate, triethyl citrate, acetyl tributyl citrate, tributyl citrate or acetyl tributyl citrate.

In certain embodiments, the isobutyrate derivative may be a C₁-C₆ carboxylic acid ester of sucrose such as sucrose acetate isobutyrate

In certain embodiments, the at least one plasticizer may be dipropylene glycol dibenzoate and/or acetyl tributyl citrate.

The at least one plasticizer may be present in an amount ranging from about 1% to about 10% by weight, more preferably from about 2% to about 9% by weight, and still more preferably from about 3% to about 8% by weight.

According to embodiments of the present invention, the nail composition also includes at least one solvent. The solvent may have a molecular weight less than or equal to 200. One or more of the solvents (and preferably all of the solvents) may have a boiling point between 55° C. and 250° C. In some embodiments, one or more of the solvents may have a boiling point between 120° C. and 250° C.

In certain embodiments, the solvent comprises at least one acetic or lactic acid ester having a C₁-C₄ alkyl group, including but not limited to, methyl acetate, ethyl acetate, ethyl lactate, n-propyl acetate, isopropyl acetate, and butyl acetate. For example, in some embodiments, the at least one solvent consists of ethyl acetate.

In certain embodiments, the solvent comprises at least one cyclic carbonate ester, including but not limited to, ethylene carbonate and/or propylene carbonate.

In certain embodiments, the solvent comprises at least one alcohol having a C₁-C₄ alkyl group.

The total amount of solvent present in the formula is preferably at least 10% by weight. Certain embodiments utilize between 10% and 90% by weight. In preferred embodiments, the solvent may be present in an amount ranging from about 40% to about 80% by weight, and more preferably from about 60% to about 80% by weight.

The composition also includes a film forming polymer, and may include two or more polymers.

In certain embodiments, the film former may comprise an acrylic polymer (homopolymer or copolymer). Specific examples of suitable primary film formers include, but are not limited to, synthetic polymers of the polycondensate type or of the free-radical type, acrylic polymers resulting from the copolymerization of monomers chosen from the esters and/or amides of acrylic acid and/or of methacrylic acid (as examples of monomers of ester type, mention may be made of methyl methacrylate, ethyl methacrylate, butyl methacrylate, isobutyl methacrylate, 2-ethylhexyl methacrylate and lauryl methacrylate. As examples of monomers of amide type, mention may be made of N-t-butylacrylamide and N-t-octylacrylamide), and acrylic polymers obtained by copolymerization of ethylenically unsaturated monomers containing hydrophilic groups, preferably of nonionic nature, such as hydroxyethyl acrylate, 2-hydroxypropyl acrylate, hydroxyethyl methacrylate and 2-hydroxypropyl methacrylate.

In certain embodiments, the film forming polymer may comprise (meth)acrylate homopolymers and copolymers, polyurethanes, polyacryls, polymethacryls, cellulosic polymers, styrene-acryl copolymers, polystyrene-polyacryl mixtures, polysiloxanes, polyethers, polyesters, urethane-acryl copolymers, cellulose acetate propionate, siloxane-urethane copolymers, polyurethane-polymethacryl mixtures, silicone-acryl copolymers, vinyl acetate polymers, and mixtures thereof.

The film forming polymer may comprise aldehyde condensation products such as arylsulfonamide formaldehyde resins, specifically toluene sulfonamide formaldehyde resin which is a condensation product of formaldehyde and toluene sulfonamide, toluene sulfonamide/epoxy resins, e.g. tosylamide and non-drying alkyd resins, acrylic polymers and copolymers, polyurethane, polyacryls, polymethacryls, styrene-acryl copolymers, polystyrene-polyacryl mixtures, poly siloxanes, polyethers, polyesters, urethane-acryl copolymers, siloxane-urethane copolymers, polyurethane-polymethacryl mixtures, vinyl acetate polymers, and mixtures thereof.

In certain embodiments, the film forming polymer may be a polymer comprising repeating units of at least one (meth)acrylate unit and vinyl copolymers. Potential acrylates copolymers include, but are not limited to, those sold under the PECOREZ® name such as, for example, PECOREZ AC 50.

In certain embodiments, the film forming polymer may be a slightly branched, hydroxyl-bearing polyester. Potential slightly branched, hydroxyl-bearing polyesters for use in the present invention include, but are not limited to, those sold under the BAYCUSAN® name such as, for example, BAYCUSAN® XP 8000.

In certain embodiments, the film forming polymer may be a polyurethane in solvents, having between about 20% and about 70% solids content. Certain embodiments may be aliphatic resins. Potential urethanes for use in the present invention include, but are not limited to, those sold under the REZE-LASTIC™ or REZE-MERIC™ name such as, for example, REZE-LASTIC™ 2140, REZE-MERIC™ 3182, or REZE-MERIC™ 3197.

The film forming polymer may comprise polyester resins formed by reacting a polyhydric alcohol with a polybasic acid, e.g., phthalic acid, such as the commercial product sold by Unitex Chemical Corporation under the name UNIPLEX 670-P, which is a polyester resin obtained by reacting trimellitic acid, neopentyl glycol, and adipic acid. (Meth)acrylic resins according to the disclosure may include copolymers of methyl methacrylate with butyl acrylate, butyl methacrylate, isobutyl methacrylate, or isobornyl methacrylate, for example, the commercial products PARALOID DM-55, PARALOID B48N, PARALOID B66, and ELVACITE 2550; copolymers of isobutylmethacrylate and butyl methacrylate, for instance, the commercial product ELVACITE 2046; and isobutyl methacrylate polymers, for example, PARALOID B67.

In some embodiments, at least one film forming polymer is selected from carboxyl functional acrylate polymers having a moderate to high glass transmission temperature (Tg) value. The “moderate to high glass transmission” as used in the context of the present invention, refers to an acrylates copolymer having Tg value ranging from about 40° C. to about 95° C., more preferably from about 60° C. to about 90° C., and more preferably from about 70° C. to about 85° C. In some embodiments, at least one film forming polymer has a relatively high acid value, such as for example an acid value (number) ranging from about 40 to about 95, preferably from about 50 to about 90, and preferably from about 60 to about 80, including all ranges and subranges therebetween. Commercially available examples of these film forming polymers include but are not limited to, for example, those sold under the ISOCRYL® name by Estron Chemicals such as, for example, Isocryl C-70 (styrene/acrylates copolymer), Isocryl N-2513, Isocryl H-60, and Isocryl H-1871.

The film forming polymer may comprise an epoxy resin. In some embodiments, the epoxy resin has a glass transition temperature (Tg) of less than about 100° C., preferably less than about 80° C. Non-limiting examples of suitable epoxy resins include tosylamide epoxy resins, such as those sold by Estron Chemical under the tradename Polytex™, e.g., E-75, E-100, and NX-55, NX-3214). Other non-limiting examples of suitable epoxy resins include aryl-sulfonamide epoxy resins.

In certain embodiments, the film former comprises a silicone-organic polymer hybrid compound.

In some embodiments, The silicone-organic polymer may comprise a silicone acrylate copolymer, which may have a glass transition temperature (Tg) of greater than 20° C. Suitable examples of silicone acrylate copolymers include silicone/(meth)acrylate copolymers. Suitable examples also include polymers derived from non-polar silicone copolymers comprising repeating units of at least one polar (meth)acrylate unit and vinyl copolymers grafted with at least one non-polar silicone chain. Non-limiting examples of such copolymers are acrylates/dimethicone copolymers such as those commercially available from Shin-Etsu, for example, the products sold under the tradenames KP-545 (cyclopentasiloxane (and) acrylates/dimethicone copolymer), KP-543 (butyl acetate (and) acrylates/dimethicone copolymer), KP-549 (methyl trimethicone (and) acrylates/dimethicone copolymer), KP-550 (tentative INCI name: isododecane (and) acrylate/dimethicone copolymer), and mixtures thereof. Additional examples include the acrylate/dimethicone copolymers sold by Dow Corning under the tradenames FA 4001 CM SILICONE ACRYLATE (cyclopentasiloxane (and) acrylates polytrimethylsiloxymethacrylate copolymer) and FA 4002 ID SILICONE ACRYLATE (isododecane (and) acrylates polytrimethylsiloxymethacrylate Copolymer), and mixtures thereof. Suitable examples also include polymers comprising a backbone chosen from vinyl polymers, methacrylic polymers, and acrylic polymers, and at least one chain chosen from pendant siloxane groups. These polymers may be sourced from various companies. One such company is Minnesota Mining and Manufacturing Company which offers these types of polymers under the tradenames “Silicone Plus” polymers (for example, poly(isobutyl methacrylate-co-methyl FOSEA)-g-poly(dimethylsiloxane), sold under the tradename SA 70-5 IBMMF). Suitable examples also include silicone/acrylate graft terpolymers, for example, the copolymers described in WO 01/32727 A1, the disclosure of which is hereby incorporated by reference. Suitable examples also include polymers comprises a backbone chosen from vinyl backbones, methacrylic backbones, and acrylic polymeric backbones and further comprises at least one pendant siloxane group. A non-limiting example of these polymers is poly(dimethylsiloxane)-g-poly(isobutyl methacrylate), which is commercially available from 3M Company under the tradename VS 70 IBM.

The at least one silicone-organic polymer hybrid compound may be a silicone vinyl acetate compound and/or a crosslinked anionic copolymer comprised of organic polymer blocks and silicone blocks, resulting in a multiblock polymer structure, or mixtures thereof. In particular, suitable examples of the silicone-organic polymer hybrid compound of the present invention include, but are not limited to, copolymers of vinyl acetate and vinyl silicone. Such copolymers may optionally further include C3-7 carboxylic acid and/or vinyl C7-45 alkyl ester monomers. Suitable examples of the silicone-organic polymer hybrid compound of the present invention include, but are not limited to, crosslinked anionic copolymers comprising at least one cross-linked polysiloxane structural unit. Specific examples include a compound having the INCI name of Crotonic Acid/Vinyl C8-12 Isoalkyl Esters/VA/Bis-Vinyldimethicone Crosspolymer which is a copolymer of Crotonic Acid, vinyl C8-12 isoalkyl esters and Vinyl Acetate crosslinked with bis-vinyldimethicone. This compound is commercially available from the company Wacker Chemie AG under the tradename WACKER BELSIL® P1101 (may also be known under the tradename WACKER BELSIL ® P101). Crotonic Acid/Vinyl C8-12 Isoalkyl Esters/VA/Bis-Vinyldimethicone Crosspolymer is also known by the technical name of Crotonic Acid/Vinyl C8-12 Isoalkyl Esters/VA/divinyldimethicone Crosspolymer.

The film former may comprise a cellulose derivative. Cellulose derivatives may include those obtained by substituting a part or all of the hydroxyl groups in the cellulose with other organic groups. Examples of such as celluloses are methyl cellulose, ethyl cellulose, propyl cellulose, butyl cellulose, acetyl cellulose, nitrocellulose, cellulose phthalate, hydroxyethyl cellulose, hydroxy propyl cellulose, cellulose acetate propionate, cellulose acetate butyrate, and cellulose acetate.

In certain embodiments, the film former comprises an alkyd resin. Preferably, the at least one alkyd resin is a polyester comprising hydrocarbon chains of fatty acids. Such resins can be obtained by polymerization of polyols and polyacids or their corresponding anhydride in the presence of fatty acids, where the fatty acids can be employed “as is” or in the form of fatty acid triglycerides or in the form of oils during the synthesis of the alkyd resin. Examples of suitable fatty acids which can be employed in the synthesis of alkyd resins include, but are not limited to, at least one of fatty acids corresponding to the formula R—COOH, in which R denotes a saturated or unsaturated hydrocarbon radical preferably having from 7 to 45 carbon atoms, preferably from 9 to 35 carbon atoms, preferably from 15 to 35 carbon atoms and preferably from 15 to 21 carbon atoms. Mention may be made of, for example, palmitic acid, stearic acid, oleic acid, ricinoleic acid, linoleic acid, linolenic acid and, in particular, capric acid. Specific examples of acceptable alkyd resins include, but are not limited to, at least one of those sold under the names “Beckosol ODE 230 70E” by Dainippon Ink & Chem (phthalic anhydride/glycerol/glycidyl decanoate copolymer in ethyl acetate at 70%), “NECOWEL 581®” (50% in soybean oil), “NECOWEL 585®” (20% in sunflower oil), “NECOWEL 580®” (20% in sunflower oil), “NECOWEL 586 N®” (50% in soybean oil), “NECOWEL EP 1161 g” (50% in soybean oil), “NECOWEL EP 1213®” (20% in oil), “NECOWEL EP 2009®” (32% in sunflower oil), “NECOWEL EP 2019®” (20% in oil), “NECOWEL EP 22750” (35% in oil), “NECOWEL EP 2329®” (34% in oil), and “NECOWEL EP 3016®” (30% in oil) by Ashland or “URADIL XP 515 AZ®” (73% in tall oil) or “URADEL XP 516 AZ®” (63% in tall oil) by DSM Resins. Beckosol ODE 230 70E (phthalic anhydride/glycerol/glycidyl decanoate copolymer in ethyl acetate at 70%) is particularly preferred.

The total amount of film forming polymer in the composition is preferably greater than or equal to 5% by weight, such as between 5% and 70%, 5% and 40%, or 5% and 30% by weight.

The composition also includes an oil having a ratio of between 2:1 and 4:1 of omega-6 fatty acids to omega-3 fatty acids. In preferred embodiments, the oil has between a 2:1 to 6:1 ratio of linoleic acid (C18:2) to alpha-linolenic acid (C18:3). In more preferred embodiments, this oil is cannabis sativa seed oil. This oil is preferably present in the composition in an amount less than or equal to 10.0% by weight. In certain embodiments, the oil is present in an amount between 0.001% and 10.0% by weight. In certain embodiments, the oil is present in an amount between 0.5% and 5.0% by weight.

In certain embodiments, the amount of the oil having a ratio between 2:1 and 4:1 of omega-6 fatty acids to omega-3 fatty acids is selected such that the ratio of the total amount of the film forming polymer to the oil, by weight, is between 5:1 and 20,000:1, and in preferred embodiments, between 5:1 and 40:1.

According to certain embodiments of the present application, compositions may optionally include at least one colorant. Suitable colorants include, but are not limited to, lipophilic dyes, pigments and pearlescent agents, and their mixtures. Any colorant typically found in nail polish compositions can be used.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescent pigments may also be included, and may be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Color additives, such as natural extracts, may also be appropriate in various embodiments. One such example is spirulina paltensis extract, although other extracts may also be appropriate.

The one or more colorants may be present in a total amount greater than 0.5% by weight, such as between 0.5% and 10.0% by weight, between 0.5% and 5.0% by weight, or between 0.5% and 3.0% by weight.

The composition may include other optional materials, such as fragrances, humectants, skin conditioning agents, surfactants, and preservatives.

### EXAMPLE 1

The composition listed in Table 1 was prepared by mixing all non-colorants until homogenous, then adding colorants. The mixing continued at an increased mixing speed until homogenous.

TABLE 1

| Material | % w/w |
|---|---|
| Film formers | 55-65% |
| Plasticizers | 5-10% |
| Solvents | 15-20% |
| Cannabis Sativa Seed Oil | 1% |
| Other Materials (Dimethicone, Photoprotectors, | 0.1-0.5% |
| etc.) | |
| Colorants | 5-15% |

Evaluative testing results are shown in Table 2.

TABLE 2

| | | Touch Time | Persoz |
|---|---|---|---|
| Shine at 20° | Shine at 60° | To Dry | Hardness (1 |
| (1 day) | (1 day) | (seconds) | day) |
| 52.5 ± 0.2 | 80.5 ± 0.1 | 50 | 43 ± 1 |

### EXAMPLE 2

The composition listed in Table 3 was prepared by mixing all non-colorant materials until homogenous, then adding colorants. The mixing continued at an increased mixing speed until homogenous.

TABLE 3

| Material | Formula 1 | Formula 2 | Formula 3 | Formula 4 | Formula 5 |
|---|---|---|---|---|---|
| Film formers | 15-25% | 15-25% | 15-25% | 15-25% | 15-25% |
| Plasticizers | 5-10% | 5-10% | 5-10% | 5-10% | 5-10% |
| Solvents | 55-75% | 55-75% | 55-75% | 55-75% | 55-75% |
| Cannabis | 0.0% | 0.5% | 1.0% | 2.0% | 5.0% |
| Sativa | | | | | |
| Seed Oil | | | | | |
| Other | 1-2% | 1-2% | 1-2% | 1-2% | 1-2% |
| Materials | | | | | |
| Colorants | 0.5-1% | 0.5-1% | 0.5-1% | 0.5-1% | 0.5-1% |

Evaluative testing results are shown in Table 4.

TABLE 4

| Test | Formula 1 | Formula 2 | Formula 3 | Formula 4 | Formula 5 |
|---|---|---|---|---|---|
| Persoz | 56 ± 1 | 50 ± 1 | 47 ± 1 | 43 ± 1 | 36 ± 1 |
| Hardness | | | | | |
| (1 day) | | | | | |

Those skilled in the art will recognize or be able to ascertain using no more than routine experimentation, many equivalents to the specific embodiments of the invention described herein. Such equivalents are intended to be encompassed by the following claims.

1. A nail enamel composition comprising:
at least one plasticizer;
at least one solvent selected from the group consisting of ethyl acetate, propyl acetate, butyl acetate, ethyl lactate, isopropanol, and propylene carbonate;
an alkyd resin;
at least one film forming polymer; and
cannabis sativa seed oil,
wherein the at least one plasticizer comprises an isobutyrate derivative.

2. The nail enamel composition according to claim 1, wherein the cannabis sativa seed oil is present in the nail enamel composition in a total amount of between 0.5 and 5.0% by weight.

3. The nail enamel composition according to claim 1, wherein the ratio of the amount of the at least one film forming polymer present in the composition to the amount of the cannabis sativa seed oil present in the composition, by weight, is between 5:1 and 40:1.

4. The nail enamel composition according to claim 1, wherein the at least one film forming polymer comprises a cellulose derivative.

5. The nail enamel composition according to claim 1, wherein the at least one film forming polymer comprises at least two polymers.

6. The nail enamel composition according to claim 1, wherein the at least one film forming polymer is present in the nail enamel composition in a total amount of between 5 and 70% by weight.

7. The nail composition according to claim 1, wherein the at least one plasticizer comprises a citric acid derivative.

8. The nail composition according to claim 1, wherein the at least one plasticizer comprises a benzoic acid derivative.

9. The nail enamel composition according to claim 1, wherein the at least one plasticizer is present in the nail enamel composition in a total amount of between 1.0 and 10.0% by weight.

10. The nail enamel composition according to claim 1, wherein the at least one solvent is present in an amount ranging from about 10% to about 80% by weight.

11. The nail enamel composition according to claim 1, further comprising at least one pigment.

12. The nail enamel composition according to claim 11, wherein the at least one pigment is present in an amount greater than 0.5% by weight.

13. A nail enamel composition comprising:
at least one plasticizer;
at least one solvent selected from the group consisting of ethyl acetate, propyl acetate, butyl acetate, ethyl lactate, isopropanol, and propylene carbonate;
an alkyd resin;
at least one film forming polymer; and
an oil having a ratio of omega-6 fatty acids to omega-3 fatty acids of between 2:1 and 4:1,
wherein the at least one plasticizer comprises an isobutyrate derivative.

14. The nail enamel composition according to claim 13, wherein the oil having a ratio of omega-6 fatty acids to omega-3 fatty acids of between 2:1 and 4:1 is cannabis sativa seed oil.

15. The nail enamel composition according to claim 13, wherein the at least one plasticizer further comprises a dibenzoate ester.

16. The nail enamel composition according to claim 13,
wherein the isobutyrate derivative is sucrose acetate isobutyrate.

17. The nail enamel composition according to claim 1,
wherein the at least one plasticizer further comprises a dibenzoate ester.

18. The nail enamel composition according to claim 1,
wherein the isobutyrate derivative is sucrose acetate isobutyrate.
